(19) United States
(12) Patent Application Publication
(10) Pub. No.: US 2006/0216330 A1
(43) Pub. Date: Sep. 28, 2006

(54) Peptides having an ace inhibiting effect

(76) Inventors: Johannes Gerardus Janssen, Vlaardingen (NL); Johannes Schalk, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/555,626

(22) PCT Filed: Apr. 23, 2004
(86) PCT No.: PCT/EP04/04386

§ 371 Date: Nov. 4, 2005

(30) Foreign Application Priority Data
May 5, 2003 (EP) 03076306.4

(51) Int. Cl. A61K 38/08; C12P 21/06; A61K 47/00

(52) U.S. Cl. 424/439; 514/17; 435/68.1

(57) ABSTRACT

Use of the pentapeptide IIAEK, the pentapeptide IPAVF and/or the hexapeptide IPAVFK and salts thereof for the preparation of an angiotensin-converting enzyme inhibitor.

### FIELD OF THE INVENTION

The invention relates to certain peptides for the preparation of an angiotensin-converting enzyme (ACE) inhibitor. The invention further relates to food products suitable for ACE inhibition and to a process for preparing such food products.

### BACKGROUND TO THE INVENTION

Hypertension or high blood pressure is considered to be one of the main risk factors for Cardio Vascular Diseases (CVD). One of the mechanisms which regulates blood pressure is the renin-angiotensin system. This is a cascade of reactions leading to the formation of angiotensin II, which has a strong vasoconstrictive and hence blood pressure increasing effect. Inhibition of one of the key enzymes in this cascade: Angiotensin I Converting Enzyme (ACE) reduces formation of angiotensin II and thus has a blood pressure lowering effect. Long term human intervention studies have shown regular intake of low amounts of ACE inhibitors reduces CVD by 25% (Gerstein et al. (2000), The Lancet 355, 253-259).

ACE-inhibitors in food products are well known. Such food products have for instance been prepared by fermentation of milk or milk products. In a placebo-controlled study, the blood pressure lowering effect of VPP and IPP in sour milk was shown in hypertensive humans (Hata, Y et al. (1996), American Journal of Clinical Nutrition 64, 767-771).

A commercially available fermented milk product, which claims to be “suitable for those with mild hypertension” is Calpis sour milk, fermented with Lactobacillus helveticus and Saccharomyces cervisiae, produced by Calpis Food Industry, Japan. Another commercially available fermented milk product is Evolus produced by Valio, Finland, which claims to be ‘the first European functional food to help lower blood pressure’. These fermented milk products are fermented with Lactobacillus helveticus (Lb. helveticus) strains. The products contain bio-active peptides (VPP and IPP) responsible for in vitro ACE inhibition, which are produced by proteolysis of caseins.

Another possibility identified in the prior art is preparation of ACE-inhibiting food products by enzymatic hydrolysis of milk proteins. WO 01/85984 describes the preparation of an ACE-suppressing composition by hydrolysis of whey protein isolate using the enzyme trypsin.

WO02/19837 describes a process for preparing a whey protein hydrolysate from a whey protein isolate substrate having ACE-inhibiting properties. The enzyme used for hydrolysis is Neutrase. The ACE-inhibiting peptides are given in table 1 of WO02/19837, where IPAVFK, IPAVF and IIAEK are not mentioned.

Pellegrini A, et al disclose in Biochimica et Biophysica Acta—General Subjects (2001), 1526 (2), pp. 131-140, that the peptide fragment IPAVFK has bactericidal properties. ACE inhibiting is not reported.

Nagaoka, S. et al, Biochemical and Biophysical Communications (2001) 281, 11-17 discloses IIAEK as a peptide having a hypocholesterimic effect. ACE-inhibition is not reported.

### SUMMARY OF THE INVENTION

It is an object of the invention to provide a food product suitable for ACE inhibition. A further object is to provide a food product that has an anti-hypertensive effect. It is another object to provide such food products having a good taste, in particular reduced bitterness. It is a further object to provide a food product that may be produced at an acceptable price. It is still a further object to provide a food product having a high concentration of ACE-inhibitor. One or more of these objects is attained according to the invention by the use of the pentapeptide IIAEK, the pentapeptide IPAVF and/or the hexapeptide IPAVFK and salts thereof for the preparation of an angiotensin-converting enzyme inhibitor.

### DETAILED DESCRIPTION OF THE INVENTION

The common one letter code is ordinarily used to describe amino-acids. IIAEK is beta-lactoglobulin position 71-75, IPAVF is beta-lactoglobulin position 78-82, and IPAVFK is beta-lactoglobulin position 78-83.

According to the invention, we have found that the pentapeptide IIAEK is stable in the human intestinal tract and that the peptides IPAVF and IPAVFK result in the formation of ACE-inhibiting peptide IPA during digestion in the human intestinal tract. The pentapeptide IIAEK, the pentapeptide IPAVF and/or the hexapeptide IPAVFK and salts thereof are therefore very suitable as an angiotensin-converting enzyme inhibitor. Preferably the angiotensin-converting enzyme inhibitor is a functional food product. Abubakar et. Al (1998), Journal of Dairy Science 81, 3131-3138 describes the antihypertensive activity of IPA in spontaneously hypertensive rats. In this article the ACE inhibition effect of IPA was shown. The food products according to the invention are suitable as food product that has an anti-hypertensive effect.

The invention provides a food product suitable for angiotensin-converting enzyme inhibition comprising an amount of 25 mg/g or more of IIAEK and/or 5 mg/g or more of pentapeptide IPAVF and/or 3 mg/g or more of the hexapeptide IPAVFK. Preferably the food product comprises an amount of 50 mg/g or more IIAEK and/or an amount of 10 mg/g or more pentapeptide IPAVF and/or an amount of hexapeptide IPAVFK of 6 mg/g or more. Preferably the food product is a protein hydrolyis product, more preferably a beta-lactoglobulin hydrolyis product.

Through optimisation of the fermentation or hydrolysis conditions, the production of the biologically active molecules IIAEK, IPAVF and/or IPAVFK may be maximised. The skilled person trying to maximise the production will know how to adjust the process parameters, such as hydrolysis time, hydrolysis temperature, enzyme type and concentration etc.

Preferably the molar yield of IIAEK, IPAVF and/or IPAVFK is high. The molar yield of IIAEK is defined as the molar amount of IIAEK produced divided by the molar amount of IIAEK fragments in the total mass of beta-lactoglobulin present in the starting material prior to hydrolysis. An analogous calculation gives the molar yield of IPAVF or IPAVFK. Note that the IIAEK sequence and the IPAVFK sequence are present in beta-lactoglobulin.

Preferred molar yield for IIAEK is 90-100%, for IPAVF it is 30-100%, more preferably 50-100% and most preferably 80-100%; for IPAVFK it is 15-100%, more preferably 30-100% and most preferably 50-100%.

For this process of hydrolysate-optimisation, the identity of the precursors of the active peptides needs to be known. However, detection and identification of the biologically active peptides in complex hydrolysates or ferments is a challenging task. Typically, just a few biologically active peptides are present at relatively low levels in a complex sample containing thousands of peptides. Traditional identification approaches employing repeated cycles of high-performance liquid chromatographic (HPLC) fractionation and biochemical evaluation are generally time consuming and prone to losses of activity. In the present work a continuous flow biochemical assay is coupled on-line to an HPLC fractionation system. The HPLC column effluent is split between a continuous flow ACE bioassay and a chemical analysis technique (mass spectrometry). Crude hydrolysates are separated by HPLC, after which the presence of biologically active compounds is detected by means of the on-line biochemical assay. Mass spectra are recorded continuously. Hence, structural information is immediately available when a peptide shows a positive signal on the biochemical assay.

Food products according to the invention are defined as products, suitable for human consumption, in which a beta-lactoglobulin hydrolysis product according to the invention was used as an ingredient in an effective amount, such that a noticeable ACE-inhibitory effect is obtained.

The food products according to the invention are preferably made according to a process involving the following steps:
(a) enzymatic hydrolysis of a whey protein substrate comprising beta-lactoglobulin resulting in a hydrolysed whey product;
(b) separation from the hydrolysed whey product of a fraction rich in pentapeptide IIAEK, the pentapeptide IPAVF and/or the hexapeptide IPAVFK;
(c) drying the fraction from step b) to obtain a solid rich in pentapeptide IIAEK, the pentapeptide IPAVF and/or the hexapeptide IPAVFK;
(d) using the solid prepared in step c) as an ingredient in the preparation of the food product.

The enzymatic hydrolysis step (a) may be any enzymatic treatment of a whey protein substrate leading to hydrolysis of beta-lactoglobulin resulting in liberation of one or more of IPAVFK, IPAVF and/or IIAEK.

The whey protein substrate may be any material that contains a substantial amount of beta-lactoglobulin. Examples of suitable substrates are milk, whey, whey powder, whey powder concentrates, whey powder isolates, or beta-lactoglobulin, etc. Preferably a substrate that has a high content of beta-lactoglobulin, such as whey protein isolate (WPI).

The enzyme may be any enzyme that is able to hydrolyse beta-lactoglobulin resulting in the liberation of one or more of IPAVFK, IPAVF and IIAEK. Examples of a preferred enzyme is trypsin. More preferably the enzyme is a mixture of trypsin and chymotrypsin.

The separation step (b) (or concentration step (b)) may be executed in any way known to the skilled person, e.g. by filtration, centrifugation or chromatography and combinations thereof. Preferably the separation step (b) is executed using an ultrafiltration (UF) and/or nanofiltration (NF) techniques. The pore size of the membranes used in the filtration step, as well as the charge of the membrane may be used to control the separation of the pentapeptide IIAEK, the pentapeptide IPAVF and/or the hexapeptide IPAVFK. The fractionation of whey protein hydrolysates using charged UF/NF membranes is described in Y. Poilot et al, Journal of Membrane Science 158 (1999) 105-114. Electrodialysis is for instance described in WO00/42066.

The drying step (c) involves drying the fraction from step b) to obtain a solid rich in pentapeptide IIAEK, the pentapeptide IPAVF and/or the hexapeptide IPAVFK. This step may be done in a conventional way, e.g. by spray drying or freeze drying.

The fraction rich in peptides prepared in step (b) is hereafter designated as ACE-fraction and the solid prepared in step (c) is hereafter designated as ACE-solid. The ACE-fraction and/or the ACE-solid may advantageously be used as an ingredient in a food product.

The food product according to the invention or food products derived therefrom may be pasteurised or sterilised.

The food products according to the invention may be of any food type. They may comprise common food ingredients in addition to the food product, such as flavour, sugar, fruits, minerals, vitamins, stabilisers, thickeners, etc. in appropriate amounts.

Preferably, the food product comprises 50-200 mmol/kg K⁺ and/or 15-60 mmol/kg Ca²⁺ and/or 6-25 mmol/kg Mg²⁺ more preferably, 100-150 mmol/kg K⁺ and/or 30-50 mmol/kg Ca²⁺ and/or 10-25 mmol/kg Mg²⁺ and most preferably 110-135 mmol/kg K⁺ and/or 35-45 mmol/kg Ca²⁺ and/or 13-20 mmol/kg Mg²⁺.

Preferably the food products are fruit juice products, dairy type products, frozen confectionary products or spreads/margarines. These preferred types of food products are described in some detail below and in the examples.

Fruit Juice Products

Examples of fruit juice products according to the invention are juices derived from citrus fruit like orange and grapefruit, tropical fruits, banana, peach, peer, strawberry, to which ACE-solids and/or ACE-fraction are added.

Dairy Type Products

Examples of dairy products according to the invention are milk, dairy spreads, cream cheese, milk type drinks and yoghurt, to which ACE-solids and/or ACE-fraction are added. The food product may be used as such as a milk type drink. alternatively flavour or other additives may be added. A dairy type product may also be made by adding ACE-solids and/or ACE-fraction to water or to a dairy product.

An example of a composition for a yoghurt type product is about 50-80 wt. % water, 0.1-15 wt. % ACE-solids, 0-15 wt. % whey powder, 0-15 wt. % sugar (e.g. sucrose), 0.01-1 wt. % yoghurt culture, 0-20 wt. % fruit, 0.05-5 wt. % vitamins and minerals, 0-2 wt. % flavour, 0-5 wt. % stabilizer (thickener or gelling agent).

A typical serving size for a yoghurt type product could be from 50 to 250 g, generally from 80 to 200 g.

Frozen Confectionery Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt. %, more preferred from 10 to 70 wt. %, for example 40 to 70 wt. %.

Ice cream will typically comprise 0 to 20 wt. % of fat, 0.1 to 20 wt. % ACE-solids, sweeteners, 0 to 10 wt. % of non-fat milk components and optional components such as emulsifiers, stabilisers, preservatives, flavouring ingredients, vitamins, minerals, etc, the balance being water. Typically ice cream will be aerated e.g. to an overrun of 20 to 400%, more specific 40 to 200% and frozen to a temperature of from −2 to −200° C., more specific −10 to −30° C. Ice cream normally comprises calcium at a level of about 0.1 wt %.

Other Food Products

Other food products according to the invention can be prepared by the skilled person based on common general knowledge, using hydrolysed beta-lactoglobulin or hydrolysed beta-lactoglobulin derived products, such as hydrolysed beta-lactoglobulin solids as an ingredient in suitable amounts. Examples of such food products are baked goods, dairy type foods, snacks, etc.

Advantageously the food product is an oil and water containing emulsion, for instance a spread. Oil and water emulsion is herein defined as an emulsion comprising oil and water and includes oil in water (O/W) emulsions and water in oil emulsions (W/O) and more complex emulsions for instance water-in-oil-in-water (W/O/W/O/W) emulsions. Oil is herein defined as including fat. Preferably the food product is a spread, frozen confection, or sauce. Preferably a spread according to the invention comprises 30-90 wt. % vegetable oil. Advantageously a spread has a pH of 4.2-6.0.

### EXAMPLES

### Example 1

Materials and Methods

High Resolution Screening-Mass Spectrometry (HRS-MS)

Biozate™, a hydrolyzed whey protein isolate obtained from Davisco Foods International (Le Sueur, Minn., USA), was analyzed for ACE inhibitory activity in a High Resolution Screening (HRS) instrument developed by Kiadis (Leiden, the Netherlands). The system consists of an HPLC, a continuous flow ACE bioassay for bioactivity detection and a mass spectrometer for chemical identification. The gradient HPLC system consisted of four Agilent 1100 series LC pumps (Waldbronn, Germany). Two pumps were used to deliver the solutions for chromatographic separation. The two remaining LC pumps were used to add make-up solutions to the LC column effluent and compensate for changes in organic modifier percentage and HPLC flow rate throughout the chromatographic run. The aqueous and organic modifier solutions which were used to perform gradient chromatography, consisted of 0% and 95% methanol, respectively. Similarly, the aqueous and organic modifier solutions, which acted as make-up phases after the chromatographic separation, consisted of 0 and 35% methanol. All solutions contained 0.05% TFA. The LC separations were carried out at room temperature on an Alltech Ultima, 2.1*250 mm, C₁₈ column, packed with 5 μm, 100 Å particles (Alltech, Amsterdam, the Netherlands), unless stated otherwise. The flow rate through the analytical column was 200 μl/min. The total flow after post-column makeup was kept constant at 1 ml/min and contained 10% acetonitrile. Bioactivity profiling of the samples was performed using 2-95% MeOH/0.05% TFA gradients. For the analysis 400 μl of a 0.5% Biozate solution (w/v) was used.

Using a three-way flow splitter, 50 μl/min of the gradient effluent was introduced into the continuous flow biochemical assay. 200 μl/min was directed towards a Micromass QTOF-micro mass spectrometer (Almere, The Netherlands), whereas 750 μl/min was lead to waste.

In the continuous flow biochemical assay, ACE inhibition was monitored via a substrate conversion based bioassay format. In the first step of the assay compounds eluting from the analytical column were mixed with the target protein ACE. The mixture was allowed to interact for 60 s. In a second step, an internally quenched fluorescent substrate, i.e. abz-FRK(dnp)P—OH, was added to the mixture and was allowed to interact with ACE for 120s. The fluorescence signal was monitored continuously at an excitation and emission wavelength of 320 and 420 nm, respectively. Compounds exhibiting ACE inhibitory activity temporarily reduced the rate of enzymatic substrate conversion and were detected as negative peaks in the biochemical readout. The substrate and enzyme solution, i.e. abz-FRK(dnp)P—OH (10 μM) and ACE (0.0375 U/ml), were dissolved in 200 mM Tris, 300 mM NaCl, 0.5% Tween at pH 7.5 and transferred into 50 ml Pharmacia superloops (Uppsala, Sweden). Both superloops were positioned in a Spark Mistral oven (Emmen, The Netherlands) and were thermostatted at 4° C. The superloops were connected to Agilent 1100 series LC pumps, which displaced the bioreagents at a flow rate of 25 μl/min. Affinity reactions were carried out in open tubular, knitted, polytetrafluoroethylene (PTFE), 0.5 mm i.d. reaction coils. The coils were positioned in a Shimadzu CTO-10AC vp oven (Den Bosch, The Netherlands), which was set at a temperature of 50° C. Fluorescence detection was performed with an Agilent 1100 series fluorescence detector (Waldbronn, Germany). Part of the column effluent was directed towards a QTOF-micro MS detector operated in the ESI positive ion full scan mode. The desolvation and source temperatures applied were 300 and 80° C. The capillary, sample cone and extraction voltages were set at 3000, 50 and 2.5 V. The cone and desolvation gas flow equaled 50 and 450 l/hr. Under these conditions most of the peptides showed considerable fragmentation, which facilitated structure elucidation. Caffeine was used as a lock mass and was added continuously at a flow rate of 10 μl/min. The mass spectrometer was calibrated daily with a diluted phosphoric acid solution.

Quantification of IIAEK, IPAVF and IPAVFK in the Biozate sample was performed on a Micromass Quattro II MS instrument operated in the positive electrospray, multiple reaction monitoring mode. The HPLC method used was similar to the one described above. The MS settings (ESI+) were as follows: cone voltage 37 V, capillary voltage 4 kV, drying gas nitrogen at 300 l/h. Source and nebulizer temperature: 100° C. and 250° C., respectively. The synthesized peptides were used to prepare a calibration line using the precursor ion 573.4 and the summed product ions 227.2 and 347.2 for IIAEK and using the precursor ion 546.3 and the summed product ions 282.2 and 433.1 for IPAVF and using the precursor ion 674.4 and the summed product ions 282.2 and 501.2 for IPAVFK.

In Vitro Gastro-Intestinal Digestion

The stability of peptides in the human gastro-intestinal tract was studied by subjecting Biozate™ to typical conditions in the stomach and small intestine. Gastric conditions were mimicked by dissolving 5.0 g Biozate™, 2.1 g NaCl, 0.1 g NaH₂PO₄, 0.45 g lipase and 2.9 g pepsin in 900 mL of Millipore Q water. The fluid was adjusted to pH 3.5 with HCl, stirred with a peddle (50 rpm) and kept at 37° C. for 60 min. Subsequently, intestinal conditions were mimicked by adding 9.0 g pancreatin to the simulated gastric fluid and adjusting the pH to 6.8 with NaHCO₃. The simulated intestinal fluid was incubated at 37° C. and continuous stirring (50 rpm) for 120 min. Samples were collected at different time points during the in vitro gastro-intestinal digestion. After collection, samples were directly heated at 95° C. for 30 min and subsequently stored at −20° C.

ACE Activity Measurement of IIAEK, IPAVF and IPAVFK

The ACE inhibition activity of IIAEK, IPAVF(K) were determined according to the method of Araujo et al. (2000) with some modifications. ACE activity with the fluorogenic substrate Abz-FRK(Dnp)P—OH was monitored in 0.1 M Tris buffer pH 7.0, containing 100 mM NaCl. Per reaction 150 μl 3.75 μM substrate, 20 μl 0.00625 U/ml ACE and 40 μl peptide sample was added per well in a white optiplate-96 microplate (Packerd Bioscience). The ACE activity was continuously followed in a Fluostar (BMG) fluorometer with 2 dispensers by measuring the fluorescence at λₑₓ: 320 nm and λₑₘ: 420 nm. As a standard captopril (1 nM end concentration) was used resulting in a 30% inhibition of the ACE activity.

Results and Discussion

HRS-MS Analysis of Hydrolyzed Samples

As a result, the important ACE inhibiting peptides found in Biozate™ were IIAEK (β-lactoglobulin, pos 71-75), IPAVF and IPAVFK (β-lactoglobulin, pos 78-83) at a concentration of 18.2, 4.1 and 2.31 mg/g, respectively. The IC₅₀ of IIAEK, IPAVF and IPAVFK were determined to be 20, 300 and 120 μM, respectively.

Milk proteins and milk protein hydrolysates are commonly known as precursors of a large range of ACE inhibitory peptides. After consumption, the proteins and peptides are subjected to various digestive enzymatic processes in the human gastrointestinal tract, which results in the release of in-vivo ACE inhibitory peptides. In order to assess the break-down of the identified bioactive peptides and the formation of novel active peptides after human consumption, Biozate™ was processed by an artificial gastro-intestinal tract, which simulated conditions typically found in the human body. At certain times samples were taken from the GIT model system. These were also analysed using the on-line HPLC-Bioassay-MS or HRS-MS system (table 1). It showed that IIAEK is of particular importance because of it's high resistance against GIT digestion and therefore has very high potentials to be a blood pressure lowering peptide. IPAVF and IPAVFK are important as precursors for the active peptide IPA. The IC₅₀ of IPA was 50 μM. From literature it is known that IPA has antihypertensive activity in spontaneously hypertensive rats (Abubakar et al., 1998)

TABLE 1
Results of example 1. Concentration of IIAEK, IPAVF,
IPAVFK and IPA from Biozate ™ digested in an
artificial human gastro intestinal tract (mg/L).

| | Original | t = 0 | t = 60 | t = 65 | t = 75 | t = 90 | t = 120 | t = 150 | t = 180 |
|---|---|---|---|---|---|---|---|---|---|
| Peptide | solution | min | min | min | min | min | min | min | min |
| IIAEK | 101 | 99 | 107 | 106 | 102 | 68 | 79 | 63 | 24 |
| IPAVF | 23 | 24 | 30 | 6 | 0 | 0 | 0 | 0 | 0 |
| IPAVFK | 13 | 26 | 32 | 3 | 0 | 0 | 0 | 0 | 0 |
| IPA | 0 | 0 | 0 | 9 | 9 | 9 | 9 | 14 | 17 |

### REFERENCES

Araujo, M. C., Melo, R. L., Cesari, M. H., Juliano, M. A., Juliano, L., Carmona, A. K. (2000) Peptidase specificity characterization of C- and N-terminal Catalytic sites of Angiotensin I-converting enzyme. Biochemistry 39: 8519-8525.

1. Use of the pentapeptide IIAEK, the pentapeptide IPAVF and/or the hexapeptide IPAVFK and salts thereof for the preparation of an angiotensin-converting enzyme inhibitor.

2. Use according to claim 1, wherein the angiotensin-converting enzyme inhibitor is a functional food product.

3. Use according to claim 1, wherein the peptide is IPAVFK and is used for the preparation of an antibactericidal product.

4. Use according to claim 1, wherein the peptide is IIAEK and is used for the preparation of a hypercholesterolimic product.

5. Food product suitable for angiotensin-converting enzyme inhibition comprising an amount of 25 mg/g or more of IIAEK and/or 5 mg/g or more of pentapeptide IPAVF and/or 3 mg/g or more of the hexapeptide IPAVFK.

6. Food product according to claim 5, wherein the amount of IIAEK is 50 mg/g or more and/or the amount of pentapeptide IPAVF is 10 mg/g or more and/or the amount of hexapeptide IPAVFK is 6 mg/g or more.

7. Process for the preparation of a food product according to claim 5, involving the following steps:
a) enzymatic hydrolysis of a whey protein substrate comprising beta-lactoglobulin resulting in a hydrolysed whey product;
b) separation from the hydrolysed whey product of a fraction rich in pentapeptide IIAEK, the pentapeptide IPAVF and/or the hexapeptide IPAVFK;
c) drying the fraction from step b) to obtain a solid rich in pentapeptide IIAEK, the pentapeptide IPAVF and/or the hexapeptide IPAVFK;
d) using the solid prepared in step c) as an ingredient in the preparation of the food product.

8. Process according to claim 7, wherein the enzymatic hydrolysis is performed using trypsin as enzyme.

9. Process according to claim 8, wherein a mixture of trypsin and chymotrypsin is used as enzyme.
